(19) United States
(12) Patent Application Publication
(10) Pub. No.: US 2010/0256344 A1
(43) Pub. Date: Oct. 7, 2010

(54) SURFACE MODIFICATION OF NANOSENSOR PLATFORMS TO INCREASE SENSITIVITY AND REPRODUCIBILITY

(75) Inventors: Mark E. Thompson, Anaheim, CA (US); Chongwu Zhou, Arcadia, CA (US); Richard J. Cote, Miami, FL (US); Fumiaki Ishikawa, Torrance, CA (US); Rui Zhang, Los Angeles, CA (US); Marco Curreli, Los Angeles, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: UNIVERSITY OF SOUTHERN CALIFORNIA, Los Angeles, CA (US)

(21) Appl. No.: 12/753,688

(22) Filed: Apr. 2, 2010

Related U.S. Application Data
(60) Provisional application No. 61/166,558, filed on Apr. 3, 2009.

(51) Int. Cl. C07C 245/20; C07C 43/205

(52) U.S. Cl. 534/558; 568/648; 977/750; 977/762; 977/847

(57) ABSTRACT

The present invention relates to various methods of sensitizing and modifying nanosensor platforms. In one embodiment, the present invention provides a method of increasing sensitivity by inhibiting oxidation of one or more 1,4-hydroquinone (HQ) molecules, functionalizing the nanosensor by using one or more diazonium molecules, creating one or more oxidized carbon groups on the nanosensor, and/or depositing one or more metal clusters on the nanosensor.

### CROSS-REFERENCE TO RELATED APPLICATIONS

The present application claims the benefit of priority under 35 U.S.C. §119(e) of provisional application Ser. No. 61/166,558, filed Apr. 3, 2009, the contents of which are hereby incorporated by reference.

### GOVERNMENT RIGHTS

This invention was made with government support under Contract No. R01 EB-008275-01 awarded by the National Institutes of Health. The government has certain rights in the invention.

### FIELD OF THE INVENTION

This invention relates to the field of biotechnology; specifically to nanosensor platforms and electrochemical surface functionalization and sensitivity.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Numerous efforts have been devoted to investigate the properties of carbon nanotubes (CNT) and to incorporate CNT into commercial products, especially in electronic devices and mechanical composites. Chemical and biological sensor devices is one of the numerous applications where CNTs are expected to significantly impact the field of research. CNTs are considered to be the ultimate candidate in the field of sensors because of the CNTs small diameters (usually 1-2 nm), the smallest diameter among various one-dimensional structured nanomaterials. In a CNT every atom of the material is located on the surface and thus every atom is in contact with the environment. Although several chemical and biological sensors using CNTs have been demonstrated in recent years, there have been few reports attempting to push the sensitivity of CNT biosensors systematically and further improvement is needed.

Similarly, other efforts to increase sensitivity of nanosensor platforms include site-selective surface functionalization. Among the approaches to selective surface functionalization, electrochemical activation is particularly popular due to the ability of independently addressing individual electrodes.[7] To be functionalized in a controlled manner, the surface of the electrodes needs to be activated or deactivated on demand so that an introduced molecule can be site-selectively immobilized. The activation and deactivation processes are achieved through a redox-active monolayer on the surface. By controlling the voltage on a designated electrode, the monolayer can be oxidized or reduced. In general, one of the two redox states will constitute the “OFF” state for the monolayer and this state will be chemically inert. The other redox state will on the other hand be reactive toward a certain chemical (“ON” state).

There have also been efforts to develop methods of covalent functionalization of nanotubes. However, most existing methods lack control over the extent of functionalization, often resulting in a saturation of the nanotube reactive sites. This uncontrolled functionalization is not desirable in biosensing since extensive functionalization would result in insulating nanotubes losing the gate dependence of the device. Functionalization of nanotube transistors can also be accomplished by using linker molecules that hydrophobically adsorb the nanotube sidewalls, such as pyrene derivatives and modified Tween 20. However, these linkers can be washed away with time and were found to be problematic with the attachment of highly charged molecules such as DNA. Therefore, a technique that allows control over the extent of covalent functionalization would be a very valuable tool for the surface modification of carbon nanotubes.

Finally, efforts have been made to further develop sensors based on field effect transistors (FETs) since they can offer direct, label free, electrical detection of analytes. Nanosensor FETs are mostly prepared using semiconducting nanowires or semiconducting single-walled carbon nanotubes (SWNTs). The general approach that has been used in these devices is to prepare FETs with the desired nanomaterial between source and drain electrodes and then coat the nanomaterial with a recognition agent designed to bind a specific biomolecule (analyte). Binding of the target analyte to the nanosensor causes a significant change in the environment surrounding the nanowires, leading to a change in the transconductance of the device. This change in transconductance is the sensing signal. This sensing signal has been shown to be correlated to the analyte concentration (mostly a logarithmic dependence) and it can be observed for analyte concentrations as low as femtomolar or even attomolar for devices based on Si NWs. While the sensitivity of Si NWs can be tuned by introducing dopants into the nanomaterial, carbon nanotubes are very difficult to dope. Thus, novel methods of boosting sensitivity in CNT devices would be highly valuable.

### SUMMARY OF THE INVENTION

Various embodiments include a method of increasing nanosensor sensitivity, comprising providing a nanosensor, inhibiting the oxidation of one or more compounds of the formula:

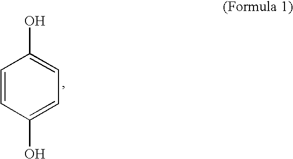

or a derivative and/or analog thereof on the surface of the nanosensor to increase sensitivity of the nanosensor. In another embodiment, inhibiting the oxidation of one or more compounds of Formula 1, or a derivative and/or analog thereof comprises attaching one or more protected redox-active molecules to the surface of the nanosensor. In another embodiment, the one or more protected redox-active molecules comprises a compound of the formula:

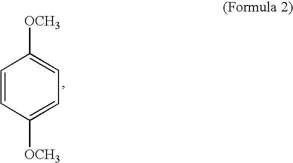

or a derivative and/or analog thereof. In another embodiment, the one or more protected redox-active molecules comprises alkyl esthers, silyl esthers, esters, carbonates, and/or sulfonates. In another embodiment, inhibiting the oxidation of one or more compounds of Formula 1, or a derivative and/or analog thereof, comprises replacing one or more compounds of Formula 1, or a derivative and/or analog thereof, with a protected redox-active molecule. In another embodiment, the nanosensor comprises a compound of the formula:

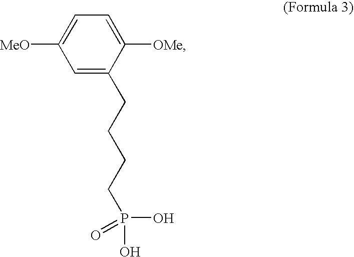

or a derivative and/or analog thereof. In another embodiment, the nanosensor comprises a compound of the formula:

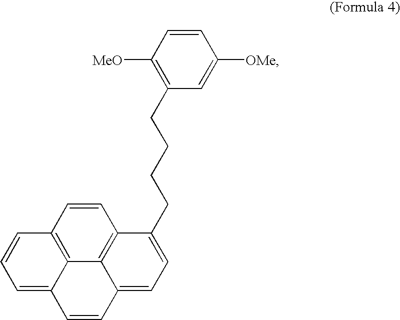

or a derivative and/or analog thereof. In another embodiment, the nanosensor comprises a self-assembled monolayer (SAM) on indium tin oxide (ITO), one or more metal oxide nanowires, and/or sidewall of a single-walled carbon nanotube (CNT) film.

Other embodiments include a method of modifying a nanotube, comprising providing a nanotube, and attaching one or more diazonium molecules to modify the nanotube. In another embodiment, the one or more diazonium molecules comprise a compound of the formula:

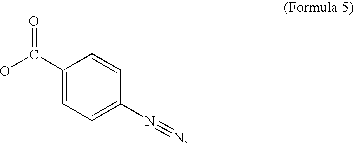

or a derivative and/or analog thereof. In another embodiment, the one or more diazonium molecules comprise a diazonium salt. In another embodiment, the one or more diazonium molecules contain a reactive functional group for bioconjugation. In another embodiment, the one or more diazonium molecules contain a carboxylic acid and/or hydroquinone functional group. In another embodiment, the nanotube comprises a sidewall of the nanotube. In another embodiment, attaching one or more diazonium molecules comprises reductive addition of the diazonium molecule.

Other embodiments include a method of increasing biosensor sensitivity, comprising providing a biosensor, and introducing one or more oxidized carbon groups on the biosensor to increase sensitivity of the nanosensor. In another embodiment, the biosensor comprises one or more single-walled carbon nanotubes (CNT). In another embodiment, introducing one or more oxidized carbon groups comprises using an oxygen plasma treatment.

Various embodiments include a method of increasing nanosensor sensitivity, comprising: providing a nanosensor, and depositing one or more metal clusters on the nanosensor to increase sensitivity of the nanosensor. In another embodiment, depositing one or more metal clusters comprises deposition of a metal precursor from a gas phase source. In another embodiment, the nanosensor comprises one or more single-walled carbon nanotubes (CNT).

Other embodiments include a method of increasing nanosensor sensitivity, comprising providing a nanosensor, and inhibiting oxidation of one or more compounds of the formula:

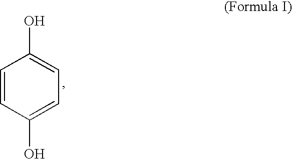

modifying the nanosensor by attaching one or more diazonium molecules to the surface of the nanosensor, creating one or more oxidized carbon groups on the nanosensor, and/or depositing one or more metal clusters on the nanosensor, to increase sensitivity of the nanosensor. In another embodiment, the nanosensor comprises one or more single-walled carbon nanotubes (CNT). In another embodiment, the nanosensor is based on a field effect transistor (FET).

Various embodiments include an apparatus comprising a nanosensor attached to the following: a protected redox-active molecule, a diazonium salt derivative molecule, an oxidized carbon species, a metal cluster, or combinations thereof. In another embodiment, the nanosensor comprises one or more single-walled carbon nanotube (CNT) and/or metal oxide nanowire.

Other features and advantages of the invention will become apparent from the following detailed description, taken in conjunction with the accompanying drawings, which illustrate, by way of example, various embodiments of the invention.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts, in accordance with embodiments described herein, electrochemically activation of surface bound 2-(1,4-dimethoxybenzene) derivatives. “X” represents the terminal group that can bind to the surface 101. The substrate can be a metal or a semiconductor material. “V” represents applied voltage.

FIG. 2 depicts, in accordance with embodiments described herein, synthesis of (A) 2-(1,4-dimethoxybenzene)-butyl phosphoric acid (“compound A”), and (B) 1-(4-(2,5-dimethoxyphenyl)butyl)pyrene (“compound B”).

FIG. 3 depicts, in accordance with embodiments described herein, cyclic voltammetry of (A) SAM of compound A on ITO-coated glass substrate and (B) a self assembled layer of compound B on CNT thin films (bucky papers).

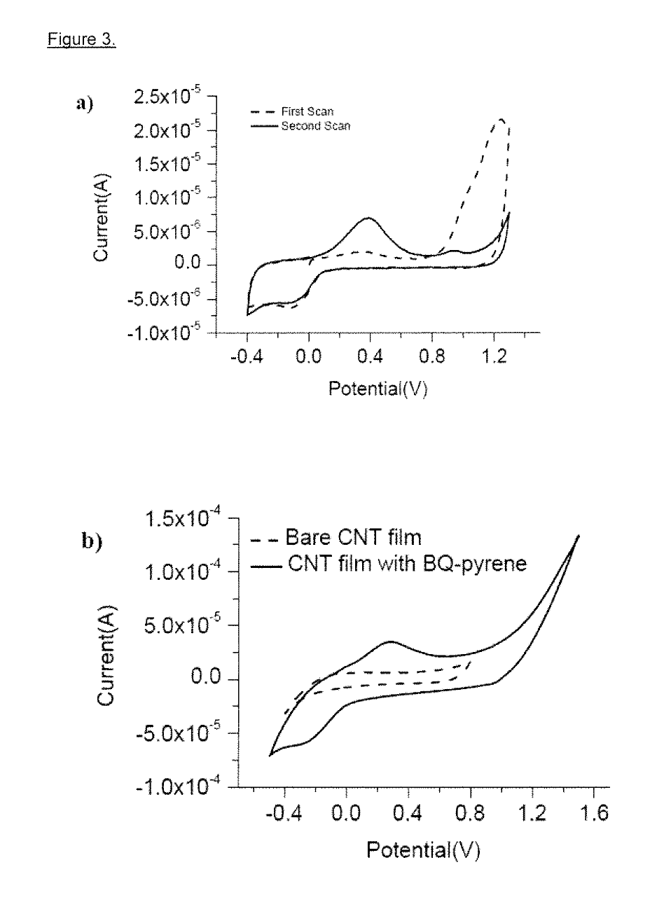

FIG. 4 depicts, in accordance with embodiments described herein, chronoamperometry of SAM of compound A on ITO-coated glass substrate.

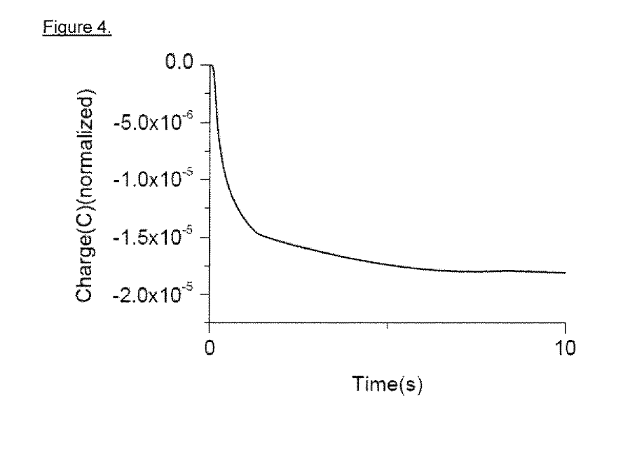

FIG. 5 depicts, in accordance with embodiments described herein, a diazonium derivative undergoes reductive addition to carbon nanotube sidewalls when the nanotubes are used as a working electrode in an electrochemical cell and the applied potential is about −250 mV versus Ag/AgCl, in 1×PBS as electrolyte.

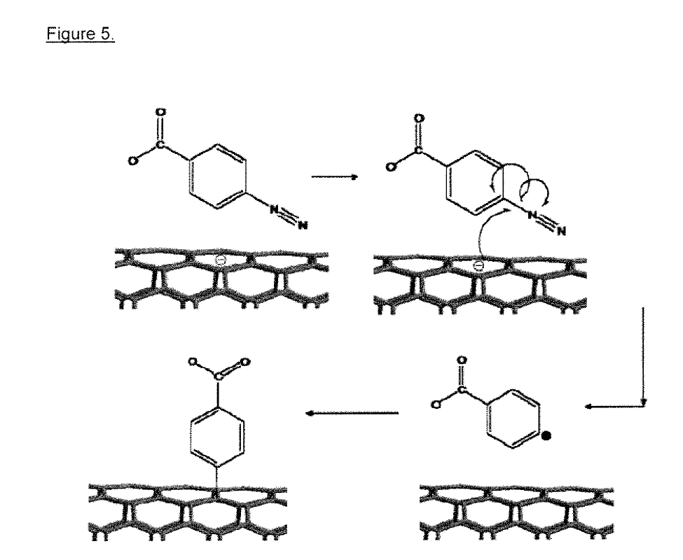

FIG. 6 depicts, in accordance with embodiments described herein, an FET based on a CNT (A) is exposed to oxygen plasma and oxidized carbon species are created on the CNT sidewalls (B). After the oxygen plasma treatment, the nanotubes are still physically present between source and drain electrodes (C).

FIG. 7 depicts, in accordance with embodiments described herein, device characteristics for a device based on a bare CNT (A) and (C) and for a device that has undergone the oxygen plasma treatment (B) and (D) before/after immobilizing streptavidin (SA).

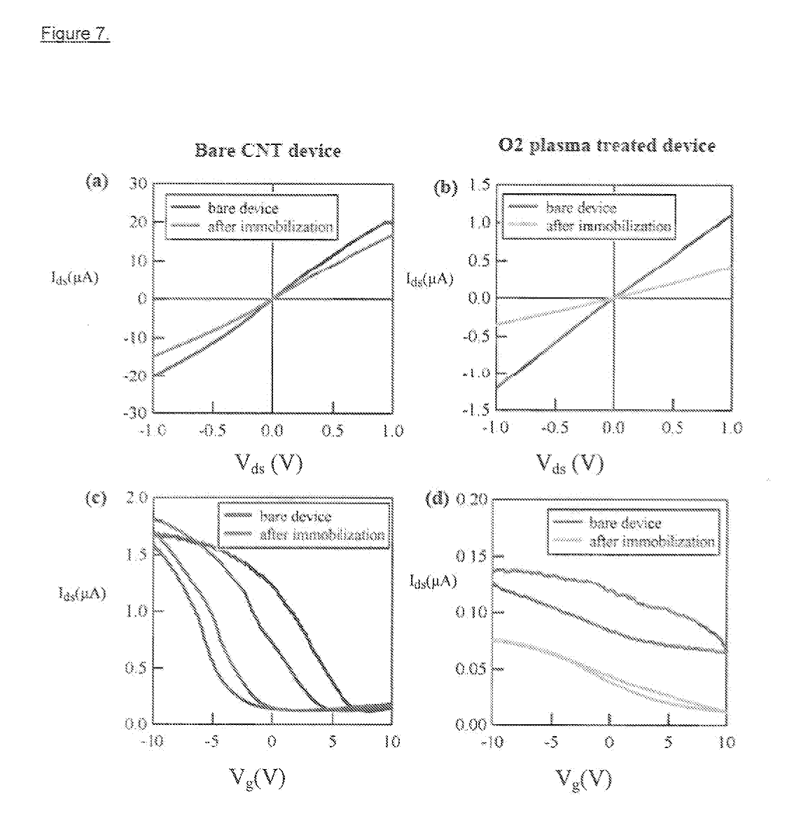

FIG. 8 depicts, in accordance with embodiments described herein, (A) schematic diagram showing metal clusters decorating the sidewalls of carbon nanotubes in a CNT FET device; (B) SEM image of a typical bare CNT device; (C) SEM image of a typical CNT device after metal cluster deposition; (D) Device characteristics (I/Vg curve) before and after metal cluster deposition. The device loses some gate dependence after metal cluster decoration.

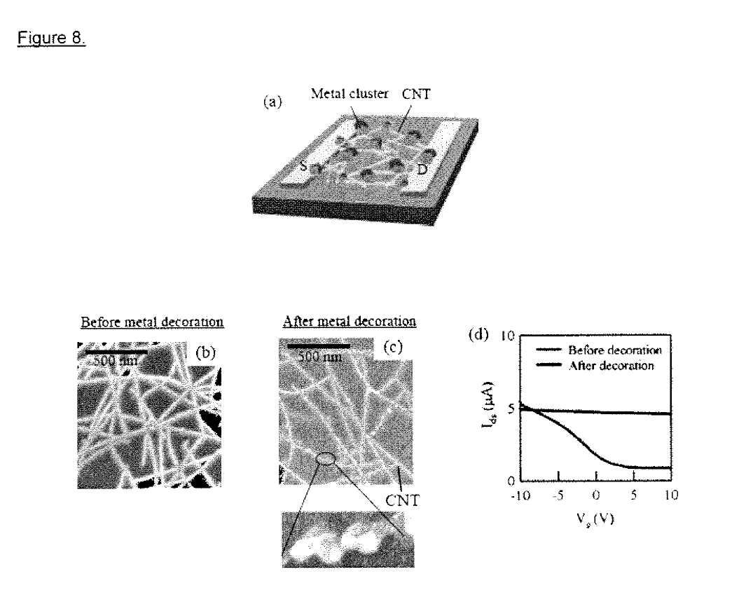

FIG. 9 depicts, in accordance with embodiments described herein, sensing traces of devices fabricated with bare CNT (A) and metal clusters decorated CNT (B). The bare CNT only shows a strong response (4% decrease in conductance) when exposed to 20 nM SA. In sharp contrast, a device decorated with metal clusters shows a 3% decrease in conductance when exposed to 100 μM SA. Clearly, metal cluster decoration improves the sensitivity by about 2000 fold.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5th ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

As used herein, “FET” means field effect transistors.

As used herein, “CNT” means carbon nanotubes.

As used herein, “SWNT” means single-walled carbon nanotubes.

As used herein, “NW” means nanowire.

“Functionalization,” as used herein, is the addition of functional groups onto the surface of a material by chemical synthesis methods.

As used herein, 2-(1,4-dimethoxybenzene)-butyl phosphonic acid is also referred to as “compound A,” a compound of the formula:

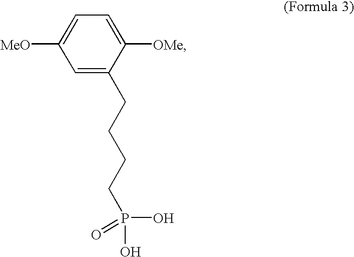

or a derivative and/or analog thereof. As used herein, 1-(4-(2,5-dimethoxyphenyl)butyl)pyrene is also referred to as “compound B,” a compound of the formula:

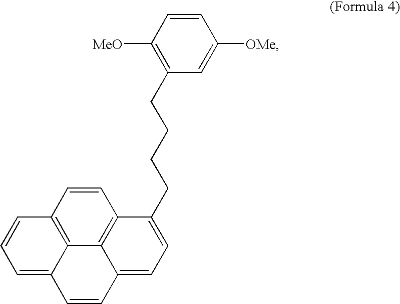

or a derivative and/or analog thereof.

As used herein, 1,4-benzoquinone is also referred to as “BQ,” and 1,4-hydroquinone is also referred to as “HQ,” and of the formula:

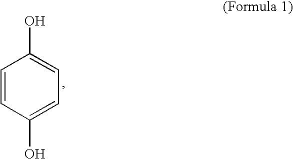

or a derivative and/or analog thereof.

As disclosed herein, the inventors have developed various methods and means of increasing sensitivity and reproducibility of nansensors. Nanosensors, such as nanowire based field effect transistors (FETs), may have a variety of commercial applications such as monitoring enzymatic activities and health monitoring, and potentially operate by detecting a variety of analytes with specificity and sensitivity. The capacity of the nanosensors to detect specific molecules may be provided via surface modification of nanosensor platforms. One example of surface modification of nanosensors is functionalization, where functional groups are added to the surface of the nanosensor by chemical synthesis methods, where the functional group added can be subjected to ordinary synthesis methods to attach virtually any kind of compound onto the surface. One approach to surface functionalization is electrochemical activiation, where electrodes are activated or deactivated on demand through a redox-active monolayer on the surface. Essentially, one of the two redox states will constitute a chemically inert and inactive state, or “OFF” state, and the other redox state will constitute a reactive state, or “ON” state. In conjunction with various embodiments described herein, methods of increasing the sensitivity of the nanosensor may include, for example, use of active molecules for electrochemically controlled site-selective functionalization, use of diazonium salts derivatives for electrochemical and controllable functionalization of carbon nanotubes, introducing oxygen plasma to create defects in carbon nanotubes, and/or coating carbon nanotubes by metal clusters.

I. Active Molecules for Electrochemically Controlled Site-Selective Functionalization

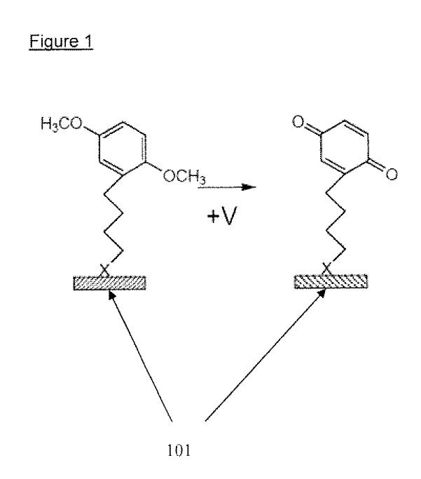

As disclosed herein, 4-benzoquinone (“BQ”)/1,4-hydroquinone (“HQ”) has been previously demonstrated as one possible redox pair that can be utilized in electrochemical controlled, site-selective surface functionalization. BQ can react with thiols, primary amines, azides, and cyclopentadienes while HQ is inactive towards all these functional groups. However, the inventors noticed that HQ derivatives can be oxidized to BQ by dissolved oxygen when placed in an aqueous solution. Additionally, the inventors observed that the rate of oxidation depends on the concentration of oxygen and pH of the aqueous solution. Therefore, over time, HQ (an “OFF” state) will be involuntarily converted to BQ (as the “ON” state) without applying any external voltage. As a result of this undesired conversion to BQ, the selectivity of this method will be greatly diminished. In response, protected redox-active molecules can be used, instead of the original unstable structure, as the “OFF” state. The protecting group can be electrochemically removed and then the “ON” state is revealed. As depicted in FIG. 1 herein, the inventors chose 1,4-dimethoxybenzene as the corresponding “OFF” state for BQ due to its availability.

As further disclosed herein, 1,4-dimethoxybenzene has been employed as a precursor in the synthesis of BQ as previously described, but not as an electrochemical “OFF” state in selective surface functionalization. 1,4-dimethoxybenzene does not react with the functional groups listed above, and moreover, it is stable in aqueous solutions in the presence of oxygen over long period of time. This chemical stability allows greater inactivity as the “OFF” state. When the inventors applied an appropriate positive voltage, this molecule can be irreversibly oxidized to BQ ('ON″ state) in aqueous media. The loss of the protecting methyl groups can be considered an electrochemical deprotection. Once 1,4-dimethoxybenzene is deprotected, the resultant BQ can be used for reactive sites for further surface reactions. 1,4-dimethoxybenzene/BQ redox pair is a versatile anchoring toos in electrochemically-induced, selective functionalization of surfaces. BQ derivatives can be immobilized on different materials by tailoring the terminal group. The inventors synthesized a 1,4-dimethoxybenzene derivative with phosphonic acid terminal, which can form a self-assembled monolayer (SAM) on indium tin oxide (ITO) and metal oxide nanowires, such as indium oxide nanowires. The inventors have also synthesized a 1,4-dimethoxybenzene derivative with a pyrene terminal, which absorbed on the sidewalls of carbon nanotube (CNT) films (bucky papers) and CNTs in the FET channel. The pyrene terminal group was chosen as a proof of concept and is not so limited as there are any number of additional terminal groups that bind to the nanotube sidewalls, such as Tween 20 (hydrophobic interaction) and diazonium derivatives (covalent binding). These and other binding groups will be considered to optimize the density of 1,4-dimethoxybenzene/BQ derivative at the surface. Cyclic voltammetry showed the irreversible oxidation peak of compound A at the first scan, and the disappearance of this peak in the second scan and the appearance of new redox peaks revealed the conversion of the head group to BQ. Compound B also showed reversible redox peak after electrochemically deprotection. The molecular coverage of SAM of compound A was determined by chronoamperometry. Compared to the BQ/HQ pair, the 1,4-dimethoxybenzene/BQ redox pair has better selectivity when used for surface functionalization of a large number of electrodes. The complete inactivity of the “OFF” state can prevent cross-contamination of electrode surfaces, especially when more than one compound is immobilized.

In one embodiment, the present invention provides a method of site-selective functionalization where active molecules are used to electrochemically control functionalization of a surface in a site-selective manner. In another embodiment, the surface may comprise metal electrodes, semi conducting surfaces, and/or nanomaterials. In another embodiment, the metal eletrodes are gold and/or platinum. In another embodiment, the semiconducting surface includes silicon and/or gallium nitride. In another embodiment, the nanomaterials comprise carbon nanotubes, metal oxide nanowires, group IV nanowires, and/or quantum dots.

In another embodiment, the present invention provides a method of using protected redox-active pairs for electrochemical controlled, site-selective functionalization. In another embodiment, one or more redox active pair comprise 1,4-hydroquinone and/or 1,4-benzoquinone.

As readily apparent to one of skill in the art, the choice of the protecting groups for the benzenediol is not limited to alkyl ether and any number of protecting groups that provide stability for both the “ON” and “OFF” states of the nansensor may also be used. Other protecting groups including silyl ethers, esters, carbonates, and sulfonates can also be used as long as they can be electrochemically removed. Similarly, it should be noted that this can not only be applied to the 1,4-hydroquinone/1,4-benzoquinone pair but also other redox-pairs with unstable “OFF” states.

II. Diazonium Salts Derivatives for the Electrochemical, Controllable Functionalization of Carbon Nanotubes for Biosensing Application

As disclosed herein, the inventors have developed a method of covalently adding functional groups by chemical syntheis methods to the surface of carbon nanotubes in sensors based on field effect transistors, using an electrochemical technique involving derivatives of diazonium salts. This technique allows controlling the extent of functionalization so that the carbon nanotubes retain their electronic properties. Many of the alternative methods of functionalization that currently exist in the field are problematic because they lack control over the extent of the functionalization, resulting in oversaturation of reactive sites on the nanotube, which in turn causes the undesirable alteration of the devices' characteristics.

As further disclosed herein, various embodiments apply to the field of biological sensing and can be used to functionalize nanotubes with a linker bifunctional molecule to immobilize biological probe molecules to the nanotubes. The method can also result in the covalent functionalization of the nanotubes and aims at attaching a small number of linker molecules to the nanotube so the electrical characteristics of the device will be unaltered. The small number of linker molecules can also be controlled by optimizing voltage (in an electrochemical cell), concentration of reactive diazonium, and time. Additionally, the electrochemical functionalization can be done in PBS as an electrolyte solution.

In one embodiment, one or more diazonium molecules are attached to the surface of the nanotube to covalently functionalize the nanotube. In another embodiment, the nanotube is a carbon nanotube in a sensor based on a field effect transistor. In another embodiment, the one or more diazonium molecules are attached to the surfact by using an eletrochemical technique with derivatives of diazonium salts. In another embodiment, the functionalizing allows control over the extent of the covalent functionalization. In another embodiment, the functionalized carbon nanotubes are prepared by following one or more of the following steps: (1) The carbon nanotube (CNT) is fabricated using semiconducting nanotubes or a mixture of semiconducting and metallic nanotubes; (2) The CNT is placed in a sample holder, where the bottom support allows applying a proper gate voltage and running current through S-D electrodes, and the top cell is filled with an electrolyte solution; (3) A linker molecule is added to the electrolyte solution at the appropriate concentration; and (4) The appropriate S-D voltage is applied for the appropriate amount of time.

As readily apparent to one of skill in the art, in conjunction with various embodiments herein, any number of molecules related to diazonium may be used to functionalize the surface of a nanotube and the invention is not in any way limited to derivatives of diazonium salts. Additionally, as readily apparent to one of skill in the art, any number of linker molecules may be used for functionalization and the invention is not in any way limited to just diazonium related molecules.

III. Oxygen Plasma to Create Defects in Carbon Nanotubes to Improve Sensitivity

As disclosed herein, the inventors used oxygen plasma to introduce defects in the form of oxidized carbon atoms on the sidewalls of carbon nanotubes (CNT), resulting in the sensitivity of biosensors on CNT field effect transistors. By creating oxidized carbon species on the sidewalls of CNT in sensor devices, the resulting devices show improved sensitivity with respect to bare CNT devices. The inventors have demonstrated this concept with the detection of streptavidin as model analyte. The oxidized carbon species was created using an oxygen plasma treatment.

In one embodiment the present invention provides a method of increasing sensitivity of biosensors by using oxygen plasma to introduce defects in the form of oxidized carbon atoms on the sidewalls of carbon nanotubes. In another embodiment, the CNT based sensor device may be prepared by one or more steps of the following procedure: (1) Catalyst islands, made of Fe₂O₃ and/or Al₂O₃, were created at pre-patterned site on a Si wafer capped with 500 nm SiO₂ following a procedure known in the literature. (2) Carbon nanotubes were grown by chemical vapor deposition (CVD) at 900° C. for 10 min. (3) Source and drain electrodes, entailing Ti (10 nm) and Au (30 nm), were then defined using photolithography. The resultant channel length and width were 4 and 40 μm, respectively. (4) Oxygen plasma was used to introduce oxidized carbon species at 10 W under 28 m Torr for 1 s.

As readily apparent to one of skill in the art, various methods are known to oxidize carbon atoms and the invention is in no way limited to oxygen plasma treatment.

IV. Metal Clusters Coating of Carbon Nanotubes as a Means to Improve Device Sensitivity

As disclosed herein, formation of nanosized metal clusters on the nanotube sidewalls has been employed as a mean to enhance sensitivity in sensor devices based on CNT. These metal clusters are formed by simple deposition of metal precursor from a gas phase source. The resulting device is stable under experimental conditions usually employed in biological sensing (aqueous solutions with acidity in 4-10 pH range and up to 1M electrolyte concentration). The size and density of the metal clusters can be easily controlled by tuning the deposition conditions. Moreover, this technique is easily applicable to full size wafers (typically 3″ or 4″ in diameter). The resulting sensors show an improvement in sensitivity by a factor of 2,000.

In one embodiment, the present invention provides a method of enhancing sensitivity in a sensor device by employing nanosized metal clusters on the nanotube sidewalls. In another embodiment, the metal clusters are formed by deposition of metal precursor from a gas phase source. In another embodiment, the sensitivity is enhanced by one or more of the following steps: (1) CNTs grown on a degenerately doped Si wafer with 500 nm SiO₂ on top via chemical vapor deposition (CVD) method with Fe nanoparticles formed from ferritin molecules as catalysts. (2) Diluted solution of ferritin in De-ionized water (D.I. water) put on the Si/SiO₂ wafer and kept for 1 h at room temperature, resulting in deposition of ferritin molecules onto the substrate. (3) The substrate then washed with D.I. water, followed by calcination in air at 700° C. for 10 min, allowing formation of Fe nanoparticles. (4) After the calcination, the substrate placed in a quartz tube is heated to 900° C. in hydrogen atmosphere, and once the temperature reached 900° C., methane (1300 sccm), ethylene (20 sccm), and hydrogen (600 sccm) flowed into the quartz tube for 10 min, which yields a CNT network on the substrate. (5) Following the growth is patterning of source-drain electrodes, done by depositing 10 nm Cr and 30 nm Au through a Cu shadow mask. The channel width and length of the resultant devices is 5 mm and 100 ˜200 μm, respectively. (6) Oxygen plasma is then performed for 1 min in order to etch unwanted CNTs while covering the channel areas with poly(methyl methacrylate) (PMMA). (7) Metal clusters then deposited onto entire devices to improve sensitivity as shown later, which was done by evaporating 3 Å Cr and 5 Å Au using an e-beam evaporator.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Active Molecules for Electrochemically Controlled, Site-Selective Surface Functionalization—Utility

Protected redox-active molecules can be employed for electrochemically controlled, site-selective surface functionalization. This technique is applicable to a large variety of surfaces including but not limited to metal electrodes (gold, platinum, etc), semiconducting surfaces (silicon, gallium nitride, etc), and nanomaterials (carbon nanotubes, metal oxide nanowires, and group IV nanowires, quantum dots, etc.).

### Example 2

### Active Molecules for Electrochemically Controlled, Site-Selective Surface Functionalization—Advantages

1,4-benzoquinone (BQ)/1,4-hydroquinone (HQ) has been demonstrated as one of redox pairs that can be utilized in electrochemical controlled, site-selective surface functionalization[1 b,c][2][4][6]. BQ can react with thiols, primary amines, azides, and cyclopentadienes while HQ is inactive towards all these functional groups. However, the inventors noticed that HQ derivatives can be oxidized to BQ by dissolved oxygen when placed in an aqueous solution. The inventors also observed that the rate of oxidation depends on the concentration of oxygen and pH of the aqueous solution. Therefore, over time, HQ (the “OFF” state) will be involuntarily converted to BQ (the “ON” state) without us applying any external voltage. As a result of this undesired conversion to BQ, the selectivity of this method will be greatly diminished. To solve this problem, protected redox-active molecules can be used, instead of the original unstable structure, as the “OFF” state. The protecting group can be electrochemically removed and then the “ON” state is revealed. The inventors choose 1,4-dimethoxybenzene as the corresponding “OFF” state for BQ due to its availability. However, it is worth noting that the choice of the protecting groups for the benzenediol is not limited to alkyl ether. Other protecting groups including silyl ethers, esters, carbonates, sulfonates and so on can also be used as long as they can be electrochemically removed. Similarly, it should be noted that this can not only be applied to 1,4-hydroquinone/1,4-benzoquinone pair but also other redox-pairs with unstable “OFF” states. 1,4-dimethoxybenzene has been employed as a precursor in the synthesis of BQ by other groups[4][6], but has never been used as electrochemical “OFF” state in selective surface functionalization. 1,4-dimethoxybenzene do not react with all the functional groups listed above, and moreover, it is stable in aqueous solutions in the presence of oxygen over long period of time. This chemical stability guarantees complete inactivity as the “OFF” state. When we applied an appropriate positive voltage, this molecule can be irreversibly oxidized to BQ (“ON” state) in aqueous media. The loss of the protecting methyl groups can be considered an electrochemical deprotection. Once 1,4-dimethoxybenzene is deprotected, the resultant BQ can be used for reactive sites for further surface reactions. 1,4-dimethoxybenzene/BQ redox pair is a versatile anchoring tools in electrochemically-induced, selective functionalization of surfaces. BQ derivatives can be immobilized on different materials by tailoring the terminal group. The inventors synthesized 1,4-dimethoxybenzene derivative with phosphonic acid terminal (compound A), which can form self-assembly monolayer (SAM) on indium tin oxide (ITO) and metal oxide nanowires. The inventors also have synthesized a 1,4-dimethoxybenzene derivative with a pyrene terminal (compound B), which absorbed on the sidewalls of carbon nanotube (CNT) films. The pyrene terminal group was chosen as a proof of concept and we are aware of other terminal groups that bind to the nanotube sidewalls, such as Tween 20 (hydrophobic interaction) and diazonium derivatives (covalent binding). These and other binding groups will be considered to optimize the density of 1,4-dimethoxybenzene/BQ derivative at the surface. Cyclic voltammetry showed the irreversible oxidation peak of compound A at the first scan, and the disappearance of this peak in the second scan and the appearance of new redox peaks revealed the conversion of the head group to BQ. Compound B also showed reversible redox peak after electrochemically deprotection. The molecular coverage of SAM of compound A was determined by chronoamperometry. Compared to the BQ/HQ pair, the 1,4-dimethoxybenzene/BQ redox pair are supposed to show better selectivity when used for surface functionalization of a large number of electrodes. The complete inactivity of the “OFF” state can prevent cross-contamination of electrode surfaces, especially when more than one compound needs to be immobilized.

### Example 3

### Active Molecules for Electrochemically Controlled, Site-Selective Surface Functionalization—Results

2-(1,4-dimethoxybenzne)-butyl phosphonic acid and 1-(4-(2,5-dimethoxyphenyl)butyl)pyrene were synthesized. Self-assembled monolayer of compound A and a self assembled layer of compound B have been formed on ITO and CNT thin films, respectively, and cyclic voltammetry was used to monitor the electrochemical activation of these surface. These surfaces can be used for selective immobilization of biological molecules terminated with thiol or primary amine. The selective functionalization will also been applied to In₂O₃ NW and SWNT based sensing devices.

### Example 4

### Active Molecules for Electrochemically Controlled, Site-Selective Surface Functionalization—Methods of Making and/or Using

2-(1,4-dimethoxybenzne)-butyl phosphonic acid (“compound A”) and 1-(4-(2,5-dimethoxyphenyl)butyl)pyrene (“compound B”) were synthesized. Monolayer of compound A was allowed to self assemble on commercial ITO-coated glass slides which were solvent cleaned and UV/O₃ treated prior to use[8]. ITO slides were first immersed into a solution of compound A (˜mM in D.I. water) for 16 hours, followed by an annealing step (140° C., N₂) for a minimum of 12 hours[9].

Carbon nanotubes films were fabricated using the vacuum filtration method previously reported by Zhang et al[10]. Compound B was dissolved by bath sonication in isopropyl alcohol. The surface of the carbon nanotubes film was flooded with the solution and left for 10 minutes. A small volume of water was then added incrementally to polarize the solution and induce interaction between pyrene and the carbon nanotube sidewalls. After 10 minutes, the solution was removed and washed away first by ethanol and then by water.

Cyclic voltammetry was performed using a custom-made Teflon cell (defined area: 0.63 cm2) with an Ag/AgCl reference electrode and a Pt wire as counter electrode. NaCl in D.I. water (0.1M) and PBS buffer were employed as supporting electrolyte for compound A and B, respectively. The molecular coverage of compound A was determined by chronoamperometry.

Site-selective surface functionalization has been previously investigated. By inducing surface reactions on demand, this technique can be used in protein micro-patterning[1][2] and electrically programmed functionalization of multielectrode devices[3]-[6].

Among several approaches to selective surface functionalization, electrochemical activation is particularly popular due to the ability of independently addressing individual electrodes[7]. To be functionalized in a controlled manner, the surface of the electrodes needs to be activated or deactivated on demand so that an introduced molecule can be site-selectively immobilized. The activation and deactivation process are achieved through a redox-active monolayer on the surface. By controlling the voltage on a designated electrode, the monolayer can be oxidized or reduced. In general, one of the two redox state will constitute the “OFF” state for the monolayer and this state will be chemically inert. The other redox state will on the other hand be reactive toward a certain chemical (“ON” state). Electrochemically controlled selective functionalization of metal[1]-[3] and semiconductor[4]-[6] surfaces has been studied by several groups. Up to now, the selective activation and deactivation of redox monolayers has been demonstrated using either a single electrode or a small number of electrodes in an array. However, the stability of the “OFF” state throughout the entire length of the experiment has to be ensured, especially when operating on an array of a large number of electrodes/devices. In other words, once the monolayer on a designated electrode is switched “OFF”, it needs to remain inactive until we would like to turn it “ON”.

1,4-benzoquinone (BQ)/hydroquinone(HQ) has been demonstrated as one of redox pairs that can be utilized in electrochemical controlled, site-selective surface functionalization[1 b,c][2][4][6]. BQ can react with thiols, primary amines, azides and cyclopentadienes while HQ is inactive towards all these functional groups. However, the inventors noticed that HQ derivatives can be oxidized to BQ by dissolved oxygen when placed in an aqueous solution. It was also observed that the rate of oxidation depends on the concentration of oxygen and pH of the aqueous solution. Therefore, over time, HQ (the “OFF” state) will be involuntarily converted to BQ (the “ON” state) without applying any external voltage. As a result of this undesired conversion to BQ, the selectivity of this method will be greatly diminished.

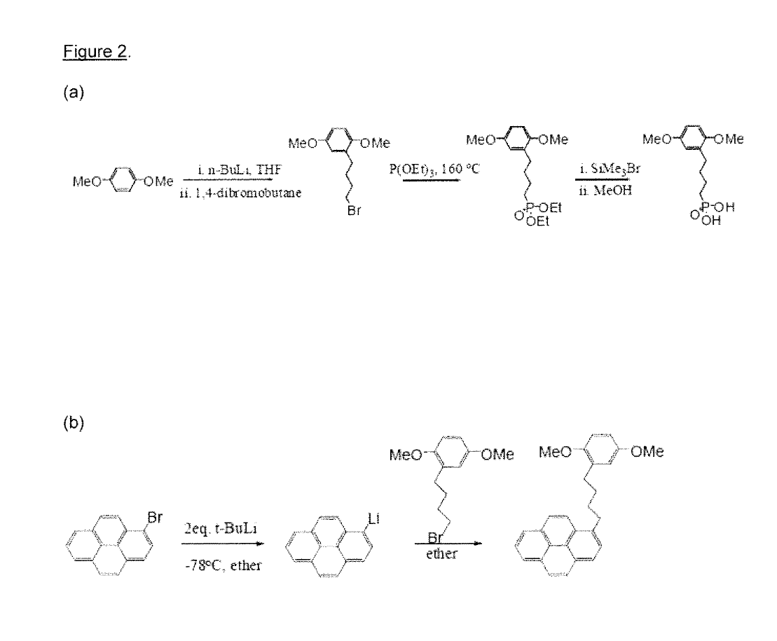

To solve this problem, the inventors propose a new structure, 1,4-dimethoxybenzene, as the “OFF” state of BQ. 1,4-dimethoxybenzene has been employed as a precursor to chemically produce BQ by other groups[4][6], but has never been used as electrochemical “OFF” state in selective surface functionalization. 1,4-dimethoxybenzene do not react with all the functional groups listed above, and moreover, it is stable in air over long period of time, which guarantees complete inactivity as the “OFF” state. When the inventors applied an appropriate positive voltage, this molecule can be irreversibly oxidized to BQ (“ON” state) in aqueous media. The loss of the protecting methyl groups can be considered an electrochemical deprotection. Once 1,4-dimethoxybenzene is deprotected, the resultant BQ can be used for reactive sites for further surface reactions. 1,4-dimethoxybenzene/BQ redox pair is a versatile anchoring tools in electrochemically induced selective functionalization and can be incorporated with different materials by tailoring the terminal group. The inventors synthesized 1,4-dimethoxybenzene derivatives with phosphonic acid terminal (A) (FIG. 2 herein) which can form self-assembly monolayer (SAM) on indium tin oxide (ITO) and metal oxide nanowires, including indium oxide nanowires. The inventors also synthesized 1,4-dimethoxybenzene derivatives with pyrene terminal (B) (FIG. 2 herein), which can absorb on thin films of carbon nanotubes (CNT) (bucky papers) and/or on CNT in the channel of FET devices. Electrochemistry experiments were performed using a custom-made Teflon cell (defined area: 0.63 cm2) with an Ag/AgCl reference electrode and a Pt wire as counter electrode. NaCl in D.I. water (0.1 M) and PBS buffer were employed as supporting electrolyte for compound A and B, respectively. Cyclic voltammetry showed the irreversible oxidation peak of compound A at the first scan at about 1.2V, and the disappearance of this peak in the second scan and the appearance of new redox peaks revealed the conversion of the head group to BQ.

Compound B also showed reversible redox peak after electrochemically deprotection. For the SAM of A on ITO-coated glass, a molecular coverage 118 Å²/molecule was determined by chronoamperometry, illustrating a fully covered surface.[8]

Compared to the BQ/HQ pair, the 1,4-dimethoxybenzene/BQ redox pair are supposed to show better selectivity when used for surface functionalization of a large number of electrodes. The complete inactivity of the “OFF” state can prevent cross-contamination of electrode surfaces, especially when more than one compound needs to be immobilized.

### Example 5

### Diazonium Salts Derivatives for the Electrochemical, Controllable Functionalization of CNT for Biosensing—Utility

The inventors have developed a method for covalently functionalizing carbon nanotubes, in sensors based on field effect transistors, using an electrochemical technique involving derivatives of diazonium salts. This technique allows controlling the extent of functionalization so that the carbon nanotubes retain their electronic properties and thus the device's characteristics are unaltered. Said method applies to the field of biological sensing. Said methods will be used to functionalize nanotubes with a linker bifunctional molecule to immobilize biological probe molecules to the nanotubes.

The method results in the covalent functionalization of the nanotubes, and aims at attaching a small number of linker molecules to the nanotube so that the electrical characteristics of the device will be unaltered. The small number of linker can be controlled by optimizing voltage (in an electrochemical cell), concentration of reactive diazonium, and time. The electrochemical functionalization can be done in PBS as electrolyte solution.

### Example 6

### Diazonium Salts Derivatives for the Electrochemical, Controllable Functionalization of CNT for Biosensing—Advantages

Various embodiments described herein allow the covalent functionalization (with a linker molecule) of nanotube FETs with preserving the device characteristics. This linker molecule can be used to immobilize biological molecules to the sidewalls of nanotubes for the purpose of configuring nanotube biosensors.

Diazonium molecules have been shown to undergo reductive addition to carbon nanotube sidewalls when the nanotubes are used as a working electrode in an electrochemical cell and the applied potential is about −250 mV versus Ag/AgCl in 1×PBS as electrolyte.

### Example 7

### Diazonium Salts Derivatives for the Electrochemical, Controllable Functionalization of CNT for Biosensing—Methods of Making and/or Using

1. CNT FETs are fabricated using semiconducting nanotubes or a mixture of semiconducting and metallic nanotubes. 2. Metallic paths can be eliminated by electrical breakdown. 3. The device is placed in a custom made sample holder entailing a PCB as bottom support (where easy to make electrical connections) and a Teflon cell on top. 4. The bottom support allows applying a proper gate voltage and running current through S-D electrodes. The top cell is filled with a PBS electrolyte solution. In this solution, the counter and reference electrodes are submerged. The underlying nanotube device is used as working electrode. 5. The linker molecule (in the form of a para-diazonium salt) is added to the electrolyte solution at the appropriate concentration. 6. The appropriate S-D and gate voltage is applied for the appropriate amount of time. During this time, the diazonium salt is electrochemically reduced and the in situ generated radical react with the carbon atoms in the nanotube. 7. The device is then washed and removed from the bottom support. 8. The nanotubes are now decorated with linker molecules bearing a second, reactive functional group useful in bioconjugation such as carboxylic acid or hydroquinone (methyl protected).

### Example 8

### Oxygen Plasma to Create Defects in Carbon Nanotubes to Improve Sensitivity—Utility

Oxygen plasma was used to introduce defects in the form of oxidized carbon atoms on the sidewalls of carbon nanotubes (CNT), resulting in an increase in the sensitivity of biosensors based on CNT field effect transistors.

### Example 9

### Oxygen Plasma to Create Defects in Carbon Nanotubes to Improve Sensitivity—Advantages

The process introduces oxidized carbon groups on the CNT sidewalls in a simple and rapid manner. These sites are used to bind capture molecules for biological analytes to the CNT sidewalls. Biosensors fabricated using this technique show an improvement in sensitivity with respect to bare CNT devices. Another advantage is the scalability of such a process to a large output of fabrication.

### Example 10

### Oxygen Plasma to Create Defects in Carbon Nanotubes to Improve Sensitivity—Results

The inventors have demonstrated that by creating oxidized carbon species on the sidewalls of CNT in sensor devices, the resulting devices show improved sensitivity with respect to bare CNT devices. The inventors have demonstrated this concept with the detection of streptavidin as model analyte. The small number of oxidized carbon species was created using an oxygen plasma treatment.

### Example 11

### Oxygen Plasma to Create Defects in Carbon Nanotubes to Improve Sensitivity—Methods of Making and/or Using

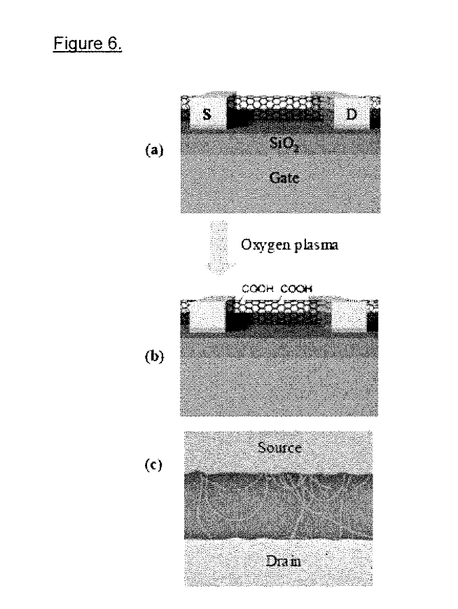

Sensor devices based on CNT were prepared by the following procedure: 1) Catalyst islands, made of Fe₂O₃ and/or Al₂O₃, were created at pre-patterned site on a Si wafer capped with 500 nm SiO₂ following a procedure known in the literature. 2) Carbon nanotubes were grown by chemical vapor deposition (CVD) at 900° C. for 10 min. 3) Source and drain electrodes, entailing Ti (10 nm) and Au (30 nm), were then defined using photolithography. The resultant channel length and width were 4 and 40 respectively. 4) Oxygen plasma was used to introduce oxidized carbon species at 10 W under 28 m Torr for 1 s. These conditions were carefully chosen so that a small number of defects could be created and the CNT were still physically present between source and drain electrodes. (FIG. 6 herein) The device characteristics for a device based on a bare CNT are shown herein and for a device that has undergone the oxygen plasma treatment. The Ids/Vds curves demonstrated that the device with oxygen plasma treatment showed larger response than the device without oxygen plasma. The Ids/Vg curves also demonstrate that the device with oxygen plasma treatment showed larger response than the device without oxygen plasma.

### Example 12

### Metal Clusters Coating of Carbon Nanotubes as a Mean to Improve Device Sensitivity—Utility

Carbon nanotubes coated with metal clusters have been used to fabricate biosensor devices. Metal cluster coating results in an increase in sensitivity with respect to bare nanotubes.

### Example 13

### Metal Clusters Coating of Carbon Nanotubes as a Mean to Improve Device Sensitivity—Advantages

Formation of nanosized metal clusters on the nanotube sidewalls has been employed as a mean to enhance sensitivity in sensor devices based on CNT. These metal clusters are formed by simple deposition of metal precursor from a gas phase source. The resulting device is stable under experimental conditions usually employed in biological sensing (aqueous solutions with acidity in 4-10 pH range and up to 1M electrolyte concentration). The size and density of the metal clusters can be easily controlled by tuning the deposition conditions. Moreover, this technique is easily applicable to full size wafers (typically 3″ or 4″ in diameter). The resulting sensors show an improvement in sensitivity by a factor of 2,000.

### Example 14

### Metal Clusters Coating of Carbon Nanotubes as a Mean to Improve Device Sensitivity—Results

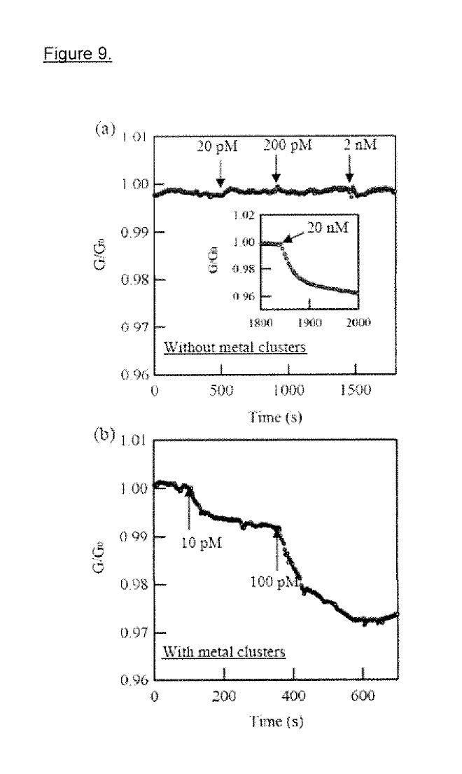

Sensor devices based on CNT were fabricated by following well established fabrication procedure followed by metal cluster deposition. The sensitivity of metal decorated devices was compared to bare CNT devices using streptavidin (SA) as a target molecule. The sensing responses of the inventors' devices are disclosed herein (FIG. 9) as plot of normalized conductance (G/Go) versus time for devices for a bare CNT device and for a metal cluster decorated device. The arrows in FIG. 9 indicate the point in time when the concentration of SA was increased to the indicated concentration. As shown in FIG. 9(a), the device without metal clusters exhibited no conductance change upon exposure to SA solutions up to 2 nM, and a conductance drop by ˜4% was observed only after exposure to a SA solution of 20 nM (FIG. 9(a) inset). The device with metal clusters, on the other hand, exhibited pronounced sensitivity, as shown in FIG. 9(b), where a conductance drop of ˜1% appeared upon exposure to SA of 10 pM, and another drop of ˜3% was observed upon exposure to 100 pM SA. Several devices with/without metal clusters were tested, and consistent results were observed. That is, devices with metal clusters exhibited higher sensitivity than devices without metal clusters by two to four orders of magnitude.

### Example 15

### Metal Clusters Coating of Carbon Nanotubes as a Mean to Improve Device Sensitivity—Methods of Making and/or Using

CNTs were grown on a degenerately doped Si wafer with 500 nm SiO₂ on top via chemical vapor deposition (CVD) method with Fe nanoparticles formed from ferritin molecules as catalysts. Diluted solution of ferritin in De-ionized water (D.I. water) was put on the Si/SiO₂ wafer and kept for 1 h at room temperature, resulting in deposition of ferritin molecules onto the substrate. The substrate was then washed with D.I. water, followed by calcination in air at 700° C. for 10 min, allowing formation of Fe nanoparticles. After the calcination, the substrate placed in a quartz tube was heated to 900° C. in hydrogen atmosphere, and once the temperature reached 900° C., methane (1300 seem), ethylene (20 sccm), and hydrogen (600 sccm) were flowed into the quartz tube for 10 min, which yields a CNT network on the substrate. Following the growth was patterning of source-drain electrodes, done by depositing 10 nm Cr and 30 nm Au through a Cu shadow mask. The channel width and length of the resultant devices were 5 mm and 100-200 μm, respectively. Oxygen plasma was then performed for 1 min in order to etch unwanted CNTs while covering the channel areas with poly(methyl methacrylate) (PMMA). Metal clusters were then deposited onto entire devices to improve sensitivity as shown later, which was done by evaporating 3 Å Cr and 5 Å Au using an e-beam evaporator.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

### REFERENCES

[1] a) Kyuwon Kim, Jaeyang Hwang, Inwoong Seo, Tae Hwan Youn and Juhyoun Kwak, Chem. Commun., 2006, 4723; b) Kyuwon Kim, Haesik Yang, Sangyong Jon, Eunkyung Kim, and Juhyoun Kwak, J. Am. Chem. Soc, 2004, 126, 15368; c) Kyuwon Kim, Minchul Jang, Haesik Yang, Eunkyung Kim, Youn Tae Kim, and Juhyoun Kwak, Langmuir, 2004, 20, 3821
[2] a) M. N. Yousaf, M. Mrksich, J. Am. Chem. Soc. 1999, 121, 4286; b) M. N. Yousaf, B. T. Houseman, M. Mrksich, Angew. Chem. 2001, 113, 1127; Angew. Chem. Int. Ed. 2001, 40, 1093; c) M. N. Yousaf, B. T. Houseman, M. Mrksich, Proc. Natl. Acad. Sci. USA 2001, 98, 5992; d) W.-S. Yeo, M. N. Yousaf, M. Mrksich, 0.1. Am. Chem. Soc. 2003, 125, 14994; e) M. Mrksich, Curr. Opin. Chem. Biol. 2002, 6, 794, and references therein
[3] Neal K. Devaraj, Pter H. Dinolfo, Christopher E. D. Chidsey, and James P. Collman, J. Am. Chem. Soc, 2006, 128, 1794
[4] Yuri L. Bunimovich, Guanglu Ge, Kristen C. Beverly, Ryan S. Ries, Leroy Hood, and James R. Heath, langmuir, 2004, 20, 10630
[5] Rosemary D. Rhode, Heather D. Agnew, Woon-Seok Yeo, Ryan C. Bailey, and James R. Heath, J. Am. Chem. Soc, 2006, 128, 9518
[6] Marco Curreli, Chao Li, Yinghua Sun, Bo Lei, Martin A. Gundersen, Mark E. Thomson, and Chongwu Zhou, J. Am. Chem. Soc, 2005, 127, 6922
[7] Insung S. Choi and Young Shik Chi, Angew. Chem. Int. Ed., 2006, 45,4894
[8] Victor M. Bermudez, Alan D. Berry, Heungsoo Kim, and Alberto Pique, Langmuir, 2006, 22, 11113
[9] a)Sergio A. Paniagua, Peter J. Hotchkiss, Simon C. Jones, Seth R. Marder, Anoma Mudalige, F. Saneeha Marrikar, Jeanne E. Pemberton, and Neal R. Armstrong, J. Phys. Chem.C., 2008, 112,7809; b) Ellen S. Gawalt, Michael J. Avaltroni, Norbert Koch, and Jeffrey Schwartz, Langmuir, 2001, 17, 5736
[10] D. Zhang, K. Ryu, X. Liu, E. Polikarpov, J. Ly, M. E. Thompson, C. Zhao, Nano Letters, Vol. 6, No. 9, 1.880 (2006).

1. A method of increasing nanosensor sensitivity, comprising:
providing a nanosensor;
inhibiting the oxidation of one or more compounds of the formula: 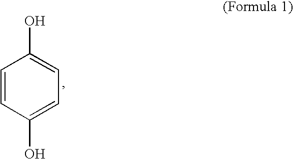
or a derivative and/or analog thereof on the surface of the nanosensor to increase sensitivity of the nanosensor.

2. The method of claim 1, wherein inhibiting the oxidation of one or more compounds of Formula 1, or a derivative and/or analog thereof comprises attaching one or more protected redox-active molecules to the surface of the nanosensor.

3. The method of claim 2, wherein the one or more protected redox-active molecules comprises a compound of the formula: 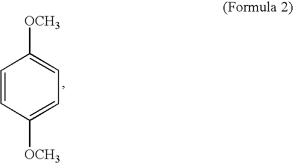
or a derivative and/or analog thereof.

4. The method of claim 2, wherein the one or more protected redox-active molecules comprises alkyl esthers, silyl esthers, esters, carbonates, and/or sulfonates.

5. The method of claim 1, wherein inhibiting the oxidation of one or more compounds of Formula 1, or a derivative and/or analog thereof, comprises replacing one or more compounds of Formula 1, or a derivative and/or analog thereof, with a protected redox-active molecule.

6. The method of claim 1, wherein the nanosensor comprises a compound of the formula: 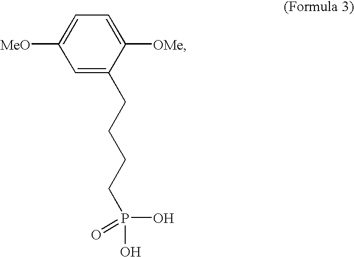
or a derivative and/or analog thereof.

7. The method of claim 1, wherein the nanosensor comprises a compound of the formula: 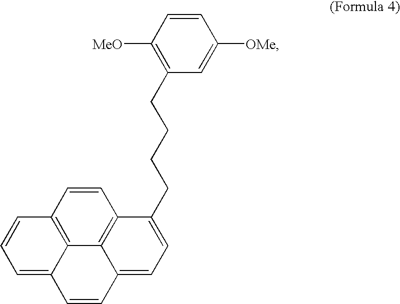
or a derivative and/or analog thereof.

8. The method of claim 1, wherein the nanosensor comprises a self-assembled monolayer (SAM) on indium tin oxide (ITO), one or more metal oxide nanowires, and/or sidewall of a single-walled carbon nanotube (CNT) film.

9. A method of modifying a nanotube, comprising:
providing a nanotube; and
attaching one or more diazonium molecules to modify the nanotube.

10. The method of claim 9, wherein the one or more diazonium molecules comprise a compound of the formula: 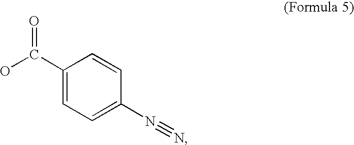
or a derivative and/or analog thereof.

11. The method of claim 9, wherein the one or more diazonium molecules comprise a diazonium salt.

12. The method of claim 9, wherein the one or more diazonium molecules contain a reactive functional group for bioconjugation.

13. The method of claim 9, wherein the one or more diazonium molecules contain a carboxylic acid and/or hydroquinone functional group.

14. The method of claim 9, wherein the nanotube comprises a sidewall of the nanotube.

15. The method of claim 9, wherein attaching one or more diazonium molecules comprises reductive addition of the diazonium molecule.

16. A method of increasing biosensor sensitivity, comprising:
providing a biosensor; and
introducing one or more oxidized carbon groups on the biosensor to increase sensitivity of the nanosensor.

17. The method of claim 16, wherein the biosensor comprises one or more single-walled carbon nanotubes (CNT).

18. The method of claim 16, wherein introducing one or more oxidized carbon groups comprises using an oxygen plasma treatment.

19. A method of increasing nanosensor sensitivity, comprising:
providing a nanosensor; and
depositing one or more metal clusters on the nanosensor to increase sensitivity of the nanosensor.

20. The method of claim 19, wherein depositing one or more metal clusters comprises deposition of a metal precursor from a gas phase source.

21. The method of claim 19, wherein the nanosensor comprises one or more single-walled carbon nanotubes (CNT).

22. A method of increasing nanosensor sensitivity, comprising:
providing a nanosensor; and
inhibiting oxidation of one or more compounds of the formula: 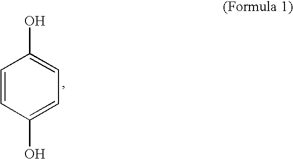
modifying the nanosensor by attaching one or more diazonium molecules to the surface of the nanosensor, creating one or more oxidized carbon groups on the nanosensor, and/or depositing one or more metal clusters on the nanosensor, to increase sensitivity of the nanosensor.

23. The method of claim 22, wherein the nanosensor comprises one or more single-walled carbon nanotubes (CNT).

24. The method of claim 22, wherein the nanosensor is based on a field effect transistor (FET).

25. An apparatus comprising:
a nanosensor attached to the following: a protected redox-active molecule, a diazonium salt derivative molecule, an oxidized carbon species, a metal cluster, or combinations thereof.

26. The apparatus of claim 25, wherein the nanosensor comprises one or more single-walled carbon nanotube (CNT) and/or metal oxide nanowire.
